(19) United States
(12) Patent Application Publication
(10) Pub. No.: US 2025/0279193 A1
(43) Pub. Date: Sep. 4, 2025

(54) TREATMENT DEVICES WITH ANTI-TAMPERING, SECURITY, AND TRANSPARENCY FEATURES

(71) Applicant: Sanmai Technologies, PBC, Sunnyvale, CA (US)

(72) Inventor: Christopher DAFT, Tucson, AZ (US)

(21) Appl. No.: 18/986,860

(22) Filed: Dec. 19, 2024

Related U.S. Application Data
(60) Provisional application No. 63/559,816, filed on Feb. 29, 2024.

(51) Int. Cl. G16H 40/40; G16H 40/67

(52) U.S. Cl. CPC G16H 40/40; G16H 40/67

(57) ABSTRACT

System and methods utilizing specialized hardware and transactions with a public blockchain protect a medical or treatment device from misuse or malicious alteration. The blockchain allows treatments to be immutably published as readable specifications or in an encrypted format. The device will not start to function without a valid interaction with the blockchain. Patient identification, treatment authorization by a healthcare provider, and payment for services are also enabled by this system.

### CROSS-REFERENCE TO RELATED APPLICATIONS

This application claims the priority benefit of U.S. Provisional Application No. 63/559,816, entitled “TREATMENT DEVICES WITH ANTI-TAMPERING, SECURITY, AND TRANSPARENCY FEATURES”, filed Feb. 29, 2024. The subject matter of this related application is hereby incorporated herein by reference.

### TECHNICAL FIELD

The invention relates to anti-tampering and security of medical devices.

### BACKGROUND

Medical and wellness devices can be tampered with or misused with harmful results. A manufacturer may wish to publish the treatments (for example, on the internet) available with its device and guarantee that its treatments will continue to be beneficial to subjects or purchasers and that the treatments have not been altered maliciously or otherwise. Patients, treating physicians, and device owners may wish to verify that the treatments provided act in the ways described by the manufacturer and have not been tampered with over time. This is a more significant issue if the devices are used in a non-clinical setting and the user downloads the treatments available. A user may inadvertently use the device for a wrong treatment (for example, a treatment not prescribed). There is a need for medical or treatment devices to have anti-tampering and security features to ensure the safety of patients or users.

### BRIEF DESCRIPTION OF THE DRAWINGS

FIG. 1 shows treatment device 101 operating in System 100, providing anti-tampering, security, and transparency.

FIG. 2 illustrates a secure treatment system 200 that can be executed on System 100.

FIG. 3 shows an exemplary method 300, where Device 101 in System 100 is used as a wellness device.

FIG. 4 shows an exemplary method 400, where Device 101 in System 100 is used as a prescription treatment device.

FIG. 5 shows an exemplary method 500 used in System 100 to update the treatment list or change the pricing.

### DETAILED DESCRIPTION

System and methods utilizing specialized hardware and transactions with a public blockchain protect a medical or treatment device from misuse or malicious alteration. The blockchain allows treatments to be immutably published as readable specifications or in an encrypted format. The device will not start to function without a valid interaction with the blockchain. Patient identification, treatment authorization by a healthcare provider, and payment for services are also enabled by this system.

In an embodiment, a secure medical or treatment device includes a secure enclave and a control block with one or more processors connected to a blockchain network via a communication module generates a request for one or more treatments to be performed by the device. The treatment device retrieves data specifying the one or more treatments from a blockchain or data availability source; requesting authorization for a selected one of the one or more treatments from the blockchain; and in response to receiving authorization from the blockchain, causing the treatment device to execute the selected treatment based on data specifying the selected treatment. In an embodiment, the treatment device includes anti-tampering functionality. Anti-tampering functionality includes physical intrusion detection, sensors to detect environmental changes (such as temperature and pressure), voltage and frequency monitoring, light detection, etc. The anti-tampering functionality can detect changes of temperatures, pressure, voltage, frequency, light etc., and create alarms if they are not within a specified range. In an embodiment, the contents of the secure enclave of the treatment device cannot be revealed or retrieved. In an embodiment, the secure enclave includes a private key which can be used to cryptographically sign communications originating from the device or to decrypt messages received. In addition to generating an alarm when tampering is detected, the contents of secure enclave may be destroyed (erased), and a device shutdown is performed. The treatment device includes user authentication to allow only authorized users to use the device in the authorized manner. The user authentication may include biometric sensors or other such functions. The treatment device communicates to a blockchain network via a communication module and includes a lightweight blockchain client.

In an embodiment, a system comprising of a p2p secure blockchain network that includes a physician's device, the treatment device described, and control device is disclosed. The physician's device (such as smartphone) connects to the p2p network to prescribe or create treatments for a user. The treatment device uses blockchain transactions to retrieve treatments lists, treatment recipes, authorizations, and pay for treatments. The control device updates treatment prices, recipes, and treatment lists on the blockchain network.

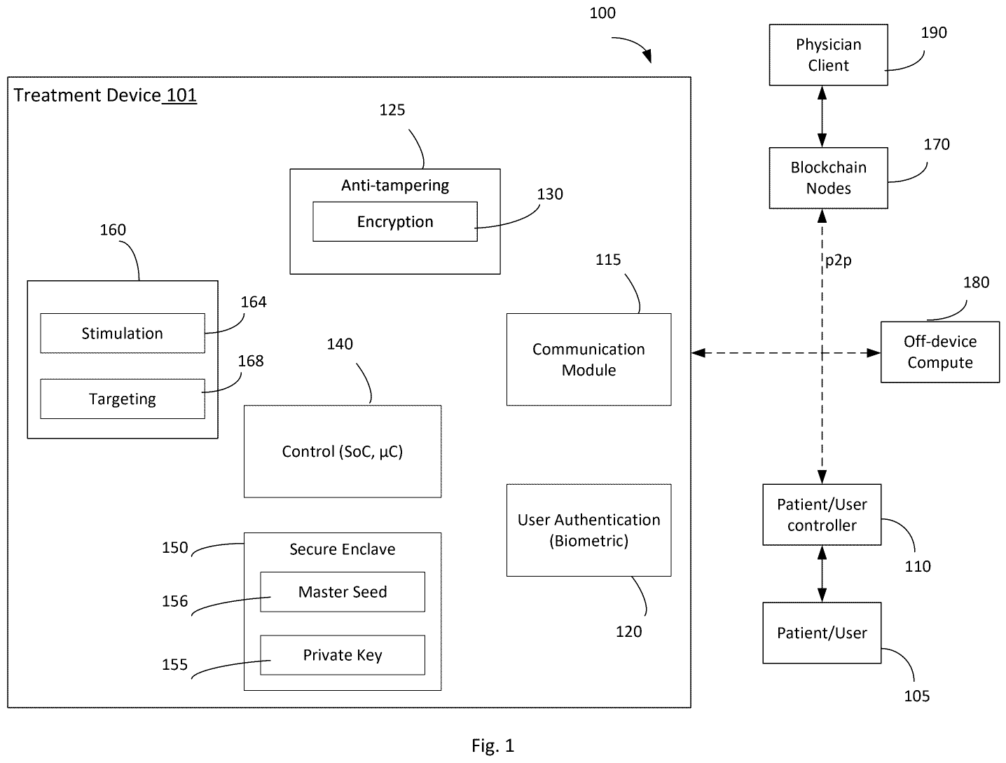

FIG. 1 shows treatment device 101 operating in System 100, providing anti-tampering, security, and transparency. In this example, treatment device 101 is a tFUS (transcranial ultrasound) device 160 with hardware and software for targeting 168 and stimulation 164. Other implementations with device 101 used for other treatments are possible. A control block 140 that contains a SoC 150 (System on Chip with one or more microprocessors) is used to control treatment, stimulation, and the overall operation of the device 101. Treatment device 101 has multiple security features. Device 101 contains logic for User (or patient) authentication 120, an anti-tampering logic 125, and a secure enclave 150. Secure enclave 150 is used for storing a private key 155; it may also store a master seed 156 that allows hierarchical deterministic (HD) wallet software to generate a large number of externally owned accounts (EOAs). Secure enclave 150 may also store biometric information generated by biometric sensors in User Authentication block 120. The secure enclave can sign messages for blockchain transactions. It also may be used for encrypting messages. Anti-tampering block 125 is used for intrusion detection and may include sensors to detect changes in device temperature, pressure, voltage, frequency, and light level. Treatment device 101 can also include specialized encryption accelerators 130 that can be used to sign or encrypt any communication to and from device 101. Communication module 115 within the device 101 handles all the communication to the device 101. Wireless protocols such as Wi-Fi or Bluetooth are used in the preferred embodiment. Wired protocols such as Ethernet or USB can also be used. Device 101 connects to Blockchain Nodes 170 via a p2p (peer-to-peer) network. Device 101 runs a light client such as WallETH and Status, allowing it to participate in the Ethereum network. Light clients have minimal storage requirements and synchronize with the network quickly since they only download block headers (rather than complete blocks). Device 101 can be implemented as a single ASIC or can be implemented as discrete devices that are integrated using a PCB, etc.

A physician with a blockchain client 190 (operating on a smartphone or other device) connects to the p2p network to prescribe treatments. Device 101 can communicate with other devices 180, servers, or off-device compute resources independent of the blockchain transactions. In the preferred embodiment, device 101 is controlled remotely by user 105 (or patient) using a controller 110. Device 101 can also be controlled by on-device UX elements (not shown) such as buttons, switches, screens, biometric sensors, etc. User authentication 120 is used for authenticating user 105. It may employ biometrics or other appropriate features to ensure that user 105 is authorized to use device 101 and that user 105 can use the device for the treatment authorized. Controller 110 may employ authentication features independent of a blockchain controls access to treatments authenticate user 105 using controller 110 to ensure that an unauthorized user is not operating the controller 110.

### Device-Level Security

The device 101 has multiple levels of security. Its blockchain interactions are one aspect; the secure enclave 150 and the anti-tampering block 125 are also important and included in some implementations.

A private key 155 and master seed 156 are written to the secure enclave 150 during manufacturing. Enclave 150 operates independently from the rest of the device 101 and cannot reveal these secrets 155. It has several functions. For example, it can sign data (such as a message) with the private key, providing a cryptographic assurance that the message originated from the device 101. This signing function is critical for blockchain transactions. It can encrypt messages which may be used outside of the blockchain transactions. It can also decrypt messages sent to the device 101, which have been encrypted with its public key.

These capabilities are helpful for firmware and software updates to the device 101. Updates can be signed by the source of the software and encrypted with device 101's public key. After the update, device 101 can sign a message confirming the update has been completed.

The functions of the anti-tampering block 125 include:

Physical Intrusion Detection: drilling, cutting, or other physical breaches using sensors that monitor the integrity of device 101's enclosure.

Environmental Sensors: to detect unusual temperature or pressure changes, indicating attempts to tamper with the device through extreme environmental conditions.

Voltage and Frequency Monitoring: monitoring for abnormal voltage or frequency levels can signify attempts to interfere with device 101's electrical operation.

Light Detection: light sensors can detect if device 101 is being opened or exposed to light unexpectedly.

Upon detecting tampering, device 101 can initiate responses like erasing data in the secure enclave, shutting down the device in a way that can be reversed only at the factory, or triggering an alarm.

Medical or wellness device 101 of FIG. 1 must be capable of accessing a smart contract blockchain. The medical or wellness device is purpose-built. It must have the ability to provide a treatment to a subject. The blockchain transactions only have value in the context of device 101; for example, deliver energy to a subject for the purpose of treatment.

Medical or wellness device 101 of FIG. 1 may produce personal health information (PHI) through its operation. For example, in the transcranial system cited above, targeting information may need to be provided to an off-device 180 computing engine. It is critically important that this PHI be utterly inaccessible to any adversary. Anti-tampering block 125 frequently contains hardware encryption accelerators 130 capable of taking even high bandwidth targeting or other information and encrypting it in real-time. The PHI can then be sent over a wireless link such as WiFi or Bluetooth, whose encryption may not be sufficiently secure for PHI.

### Blockchain Use for Device Safety

User 105 safety is guaranteed by several factors beyond the hardware aspects shown in FIG. 1.

First, the treatment hardware does not store treatment specifications. Every time the device is used, treatment specification(s) must be obtained from the blockchain.

Second, a useful characteristic for medical devices is that blockchains store data immutably. It is impossible to alter transaction or smart contract data after the chain has finalized the transaction. This means the stored data, which may be treatment specifications, cannot be altered by malicious parties or inadvertently. Subsequent attempts at alteration would be evident to anyone with access to a block explorer or a full node.

A third advantage of blockchains is that Ethereum (and many others) are designed to be decentralized. The validators, whose function is to propose and evaluate the correctness of new blocks, are numerous and geographically dispersed. For example, in early 2024, the Ethereum network had over 600,000 computers acting as validators. It also has 6,000 full nodes enforcing network rules. This level of decentralization makes it difficult even for state actors to disrupt the orderly functioning of this blockchain.

With a traditional cloud-based solution, server failure is an ever-present concern, even with fail-over arrangements. In contrast, the decentralized blockchain approach, where the smart contracts run in parallel on every Ethereum full node, is far more reliable, as evidenced by its uptime record.

Fourth, participants do not need to reveal their identities to use the network, a component of the patient privacy guarantees we seek.

### Relevant Characteristics of the Ethereum Blockchain

The present disclosure uses features available on the Ethereum blockchain but can be implemented on other smart contract blockchains. Ethereum has two types of accounts: Externally Owned Accounts (EOAs) and Smart Contract accounts. A private key controls an EOA and cannot store arbitrary data. It is limited to sending and receiving transactions, which can include information in the transaction's Data field. As its name suggests, it can also hold balances of Ether, the network's internal currency.

In contrast, a Smart Contract account can store arbitrary data. When a smart contract is executed as part of a transaction, the Data field of the transaction can contain encoded function calls and parameters for the smart contract. The smart contract can use this information to update its state on the Ethereum blockchain. These state data are persistent, remaining stored on the blockchain as long as the network exists. Alternatively, these data may be stored on other Data Availability providers controlled by the blockchain, such as Celestia, Avail or EigenDA.

### Smart Contract Functions

The smart contract arbitrates policy between the devices, physicians, patients, and treatment records. Characteristics of smart contracts allow for the safety and transparency goals to be met. In blockchains, a smart contract is a self-executing system with the terms of the agreement being directly written into lines of code. The code and agreements therein exist across a distributed, decentralized blockchain network. Smart contracts allow transactions and agreements to be carried out among disparate, anonymous parties without a central authority, legal system, or external enforcement mechanism.

Once deployed, they operate automatically without the need for an intermediary. When predefined conditions are met, the contract executes the corresponding contractual clauses. Once deployed to the blockchain, the contract cannot be altered, ensuring high security and trustworthiness. The contract output is validated by all nodes in the blockchain, making it tamper-proof and transparent.

Smart contracts are written in programming languages specific to blockchain platforms, like Solidity for Ethereum. The contract is deployed to the blockchain, which resides at a specific address. The contract automatically executes its terms when pre-set conditions, such as receiving a payment, are triggered. Based on their programming, they can automate processes, execute transactions, or enforce rules. Examples include paying for wellness treatments, registering a new treatment, or authorizing a device to perform a treatment.

The smart contract in this invention knows the complete list of published recipes. These data are stored in the smart contract's state or potentially as “blobs” on a layer-2 data availability layer. The smart contract is immutable once deployed, but upgrades can be handled by deactivating the older smart contract with a transaction, after which it may forward requests to a newly deployed new smart contract. While this approach allows the controlling entity to upgrade the system, all such upgrades are publicly visible as long as the chain functions. This provides transparency guarantees for physicians and patients, even in the face of the acquisition of the controlling entity and subsequent changes in corporate policies.

### System Operation

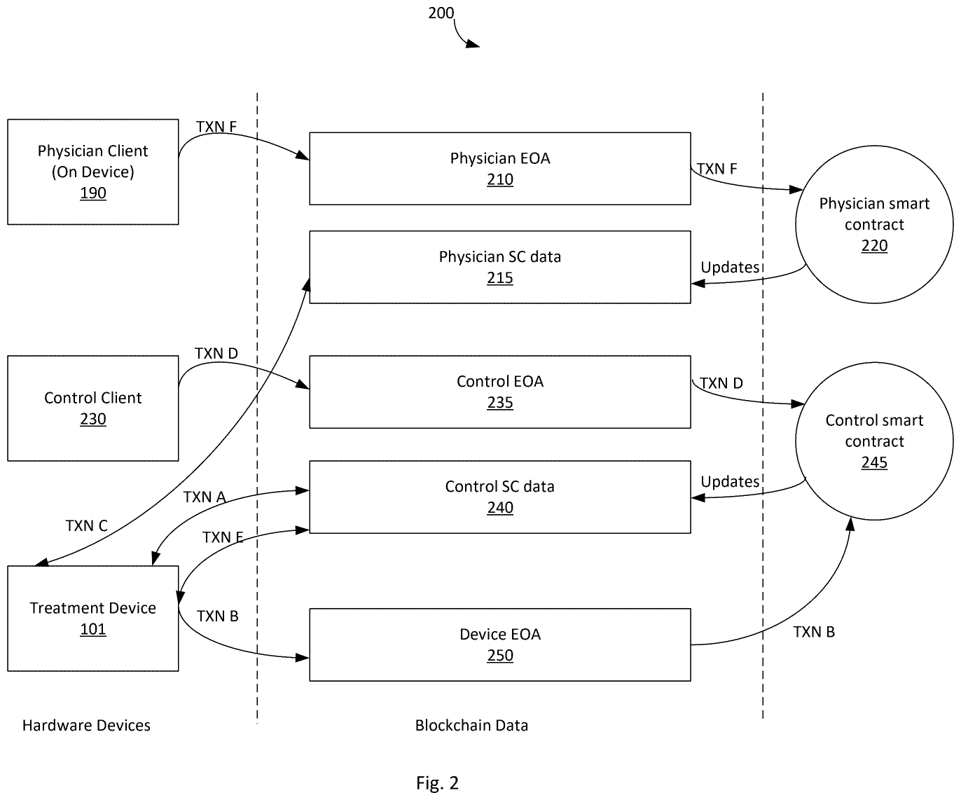

FIG. 2 illustrates a secure treatment system 200 that can be executed on System 100. Referring to FIG. 2, three categories are shown: the hardware needed, data on the blockchain, and smart contract programs (executed by EVM or Ethereum Virtual Machine), which are also stored immutably on the blockchain. The physician's activities and the control functions can be accomplished using phones 190 or computers which the control client 230 may run on. However, for the system to work, it must include a treatment device 101, such as a transcranial ultrasound system. Referring to FIG. 2, note that funds transfer is unimportant in several methods disclosed here, and the amounts transferred can be minimal. Also, “calls,” which merely query the state of the blockchain but do not change it, do not require funds. The client software may execute these.

### Unique Patient ID

Before prescribing the use of the device, a physician will have a consultation with patient 105. During this interaction, the physician generates a unique patient 105 ID (unique identification), which is entered into the device. This ID allows the patient to be identified by the physician. Alternatively, if a patient cannot meet with the physician, the ID could be transmitted to them via secure chat during a video call.

### Treatment Recipes

Definitions of treatments, referred to as “recipes,” are immutably posted as transactions to the “control” smart contract's state. Such recipes are directions to device 101 specifying aspects of the treatment, such as:
Structures to be treated.
Center frequencies of stimulation.
Length of stimulation waveforms.
Quiescent times between stimulation events.
Number of stimulation events in complete treatment.
Closed-loop parameters linking the timing of stimulation events to biomarkers measured, for example, by electroencephalography or magnetoencephalography.

### Transaction Types

Several types of transactions occur in the secure treatment system 200. These types of transactions are labeled in FIG. 2 as TXN followed by a “letter” and are defined below.
A. Device requests treatment list, treatment characteristics, and prices.
B. Device pays for a wellness treatment or a prescribed treatment.
C. Device checks whether a prescribed treatment has been authorized.
D. Controlling entity-a company or a Distributed Autonomous Organization-updates the state of the controlling smart contract, for example, to add treatments or change prices.
E. Device requests treatment recipe
F. Physician authorizes a prescription treatment.

Transaction A is a query (usually known as a “call”) about the state of the blockchain. It does not update the blockchain and can be done locally without expending gas. The device needs to run a light blockchain client to support transaction A. The term “gas” here means a quantity of a currency which is paid to the network to cause it to execute a smart contract. The amount of gas required for such EVM activity depends on the complexity of the smart contract. On the Ethereum chain, gas is denominated in “gwei,” a unit which equals 10-9 Ether. The arrow linking device 101 and Control SC data 240 indicates that the call is inquiring about the state of data 240, part of which is the available treatments, their characteristics, and their prices.

Transaction B is a payment from an EOA 250 on the device 101 to the smart contract. The device possesses several EOA addresses as well as client software to access the blockchain. The transaction's Data field includes an identifier for the treatment. Upon receipt of the payment, the smart contract 245 sends back a standard Ethereum transaction receipt, in which the “success” status code authorizes the treatment. Unlike the “call,” of Transaction A, Transaction B alters the state of the control smart contract and therefore requires gas as well as the treatment payment.

In Transaction C, the device 101 checks for authorization for a prescribed treatment. Like Transaction A, this is a “call” made to the blockchain querying the state of the Physician smart contract 220.

Transaction D causes an update of the main smart contract data 240 controlling the behavior of all treatment devices 101. For example, this type of transaction can allow for publishing a new treatment, or it can change treatment pricing. Since this changes the smart contract's state, it is a transaction from the controlling entity's EOA 235 to the smart contract 245. Note that while this alters the status of the smart contract 245, all prior state of the smart contract 245 remains immutably part of the blockchain indefinitely. This allows anyone with a block explorer or Ethereum node to check whether tampering occurs with the recipes or other aspects of the treatment control system.

Transaction E is also a “call” within the client running on the device 101. It queries the state 249 of the controlling smart contract 245 to retrieve a treatment recipe.

In Transaction F, the Physician EOA 210 sends a transaction to the Physician smart contract 220, changing its state 215 via the Data field in the transaction, which encodes smart contract function call information. This state change authorizes a certain patient to receive a particular treatment type one or more times, on a prescribed schedule.

### System Participants

An implementation of the roles of each participant shown in FIG. 2 is as follows:

Physician EOA 210 and Ethereum client: responsible for initiating transactions executed by the Physician smart contract 220. Real-life physicians can initiate such transactions using an Ethereum client from their phone or computer 190.

Physician smart contract 220: This is an on-chain object containing the state 215 of authorized treatments and providers. It can be analogized to an instance of a class in traditional software engineering. The instance contains both the treatment and provider data as well as executable code. Its executable methods are activated upon receipt of a transaction at its Ethereum address, which can alter its stored data 215. For example, a transaction from a physician's client may authorize a patient's device 101 to provide a particular treatment.

Controlling organization EOA 235 and Ethereum client 230: This is responsible for initiating transactions that trigger the execution of the Central smart contract 245, causing policy changes to its state 240.

Central smart contract 245: This on-chain object contains data and executable code, like the instance of the physician smart contract 220 class. Here its data 240 includes treatment recipes, and characteristics such as price and whether usage requires a prescription. Its internal executable functions manipulate such data upon receipt of transactions to its address.

Device EOA 250: The device hardware includes Ethereum addresses in its HD wallet to pay the central smart contract for treatments.

Device Light Client with HD wallet: The device 101 can create many addresses using a hierarchical deterministic (HD) wallet structure. With an HD wallet, only a master seed is needed to create a set of addresses. The master seed is encoded in the device during its manufacture. This means that the user of a device does not disclose their identity to the network when the device makes blockchain transactions.

### Device Use Cases

Device 101 in System 100 can be used by user 105 as a wellness device or for providing prescription treatment. The main distinction is that an authorization provided by physician client 190 is required when the device is used for prescription treatment. The transactions between device 101 and blockchain nodes 170 and within the blockchain network are invisible to user 105.

### Wellness Treatment

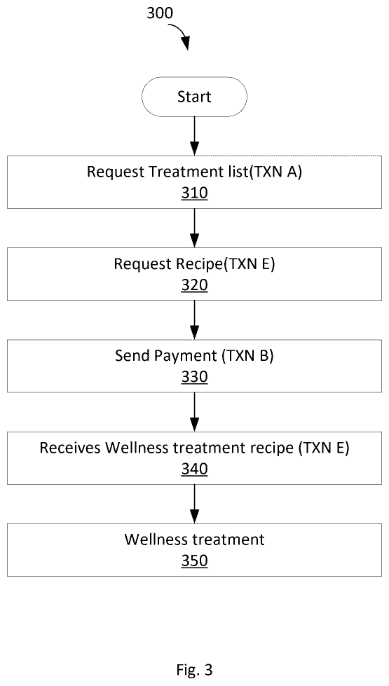

FIG. 3 shows an exemplary method 300, where Device 101 in System 100 is used as a wellness (non-prescription) device. Referring to FIG. 3, in Operation 310, user 105 requests a list of available wellness treatments. User 105 can make the request using controller 105 requesting that Controller 110 and User Authentication 120 in Device 101 authenticate user 105. For example, biometrics may be used. Device 101 ensures that user 105 is authorized and has no legal, age, business, or other restrictions. Device 101, using communication module 115, requests the wellness treatment list from Blockchain nodes 170 using the p2p network. This request is shown as Transaction (TXN) A in FIG. 2. This “call” to the blockchain incurs no cost. System 100 returns the authorized wellness treatment list to be displayed to user 105. For example, the list may be displayed as a selectable list or menu on Controller 110.

In Operation 320, user 105 requests a wellness treatment recipe. For example, user 105 selects the treatment recipe from the list displayed in Operation 310. Recipes are directions to device 101 specifying aspects of the treatment, such as:
Structures to be treated.
Center frequencies of stimulation.
Length of stimulation waveforms.
Quiescent times between stimulation events.
Number of stimulation events in complete treatment.
Closed-loop parameters linking the timing of stimulation events to biomarkers measured, for example, by electroencephalography or magnetoencephalography.

This request is displayed as Transaction (TXN) E in FIG. 2. This “call” to the blockchain incurs no cost.

In Operation 330, payment for the treatment recipe selected is sent. This is shown as Transaction (TXN) B in FIG. 2. The two components of the cost of TXN B are the gas fee to interact with the control smart contract 245, and the payment for the treatment.

In Operation 340, Device 101 receives authorization to perform the requested wellness (non-prescription) treatment. Device 101 receives the receipt of payment sent in Operation 330. An appropriate message is displayed to User 105 on Controller 110. The message may include text indicating that the requested wellness treatment is available. The message may also include detailed instructions to user 105 on how to position and use device 101.

In Operation 350, user 101 positions Device 101 appropriately and performs the requested wellness treatment.

### Prescription Treatment

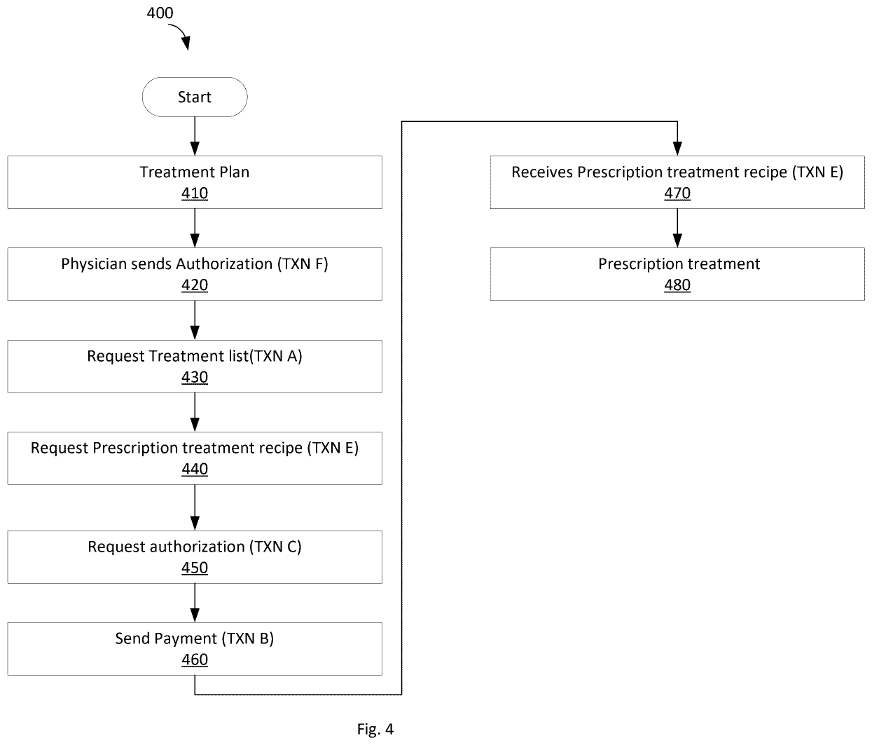

FIG. 4 shows an exemplary method 400, where Device 101 in System 100 is used as a prescription treatment device. Referring to FIG. 4, in Operation 410, Patient (or User) 105 and Physician coordinate on a treatment plan. Typically, this operation occurs off the blockchain network. Physicians (or healthcare providers) also generate a unique Patient 105 ID.

In Operation 420, the Physician uses their client 190, operating on a smartphone, computer, etc., to send an authorization transaction from their EOA for the prescription to the Physician smart contract. This is shown as Transaction (TXN) F in FIG. 2. The cost of this transaction is minimal, since only the gas to interact with the Physician smart contract 220 needs to be paid; there are no transfer of funds.

In Operation 430, user 105 requests a list of available prescription treatments. User 105 can make the request using controller 105. Controller 110 and User Authentication 120 in Device 101 authenticate user 105. For example, biometrics may be used. Device 101 ensures that user 105 is authorized and has no legal, age, business, or other restrictions. Device 101, using communication module 115, requests the wellness treatment list from Blockchain nodes 170 using the p2p network. This request is shown as Transaction (TXN) A in FIG. 2. This “call” to the blockchain incurs no cost. System 100 returns the authorized prescription treatment list to be displayed to user 105. For example, the list may be displayed as a selectable list or menu on Controller 110.

In Operation 440, user 105 requests a prescription treatment recipe. For example, user 105 selects the treatment recipe from the list displayed in Operation 430. Recipes are directions to device 101 specifying aspects of the treatment, such as:
Structures to be treated.
Center frequencies of stimulation.
Length of stimulation waveforms.
Quiescent times between stimulation events.
Number of stimulation events in complete treatment.
Closed-loop parameters linking the timing of stimulation events to biomarkers measured, for example, by electroencephalography or magnetoencephalography.

This request is displayed as Transaction (TXN) E in FIG. 2. This “call” to the blockchain incurs no cost.

In Operation 450, user 105 requests authorization (sent by the physician in Operation 420) for the prescription treatment recipe requested in Operation 440. The authorization is saved in Device 101. This is shown as Transaction (TXN) C in FIG. 2. This “call” to the blockchain incurs no cost.

In Operation 460, payment for the selected prescription treatment recipe is sent. This is shown as Transaction (TXN) B in FIG. 2, which also requires payment of gas fees because it changes the state of smart contract 245. Device 101 receives the receipt of the payment it sent.

In Operation 470, Device 101 receives authorization to perform the requested prescription treatment by querying the state of physician smart contract 220. An appropriate message is displayed to User 105 on Controller 110. The message may include text indicating that the requested prescription treatment is available. The message may also include detailed instructions to user 105 on how to position and use device 101.

In Operation 480, user 101 positions Device 101 appropriately and performs the requested prescription treatment.

### Treatment List or Pricing Change

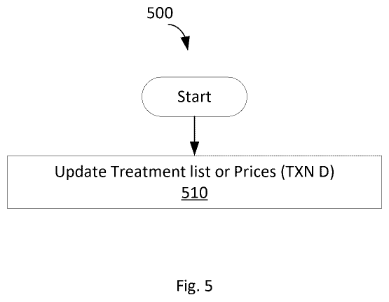

FIG. 5 shows an exemplary method 500 used in System 100 to update the treatment list, change the pricing of treatments, or otherwise make administrative changes to the behavior of the overall system as reflected in control smart contract data 240. Referring to FIG. 5, in Operation 510, the controlling organization sends a transaction from its EOA to the central smart contract specifying the needed changes (i.e., treatment list changes or treatment prices). This is shown as Transaction (TXN) D in FIG. 2. The changes can be specified for wellness and prescription treatments. This transaction only incurs gas fees, since no transfer of funds is needed to update the state of contract 245. Only the blockchain network need be paid to run the EVM to make the state changes.

### Treatment Recipe Encryption

The ethical behavior of the controlling organization can be enforced by several structures implemented. A DAO (decentralized autonomous organization) with on-chain governance fits well with this blockchain-oriented solution. A traditional corporate structure, a Public Benefit Corporation, or a non-profit may also be considered.

A prime goal of this invention is to enforce the immutability of the treatment recipes and make it easy for whistle-blowers with nothing more than a web browser or a blockchain client on their phone to identify that a recipe has been changed. However, for corporate reasons, some recipes might need to be encrypted. They would be published in encrypted form in the smart contract state. Tampering with the encrypted data would still be obvious to a whistle-blower.

Referring to FIG. 2, to support this, the secret key 155 in the device's secure enclave 150 would be used to decrypt the published information. This encryption would be independent of the blockchain operations and could use a public/private key pair or a shared secret.

The encryption could be per device, in which case a recipe encrypted for each device would need to be stored in the control smart contract data 240. This approach will be practical with cheap off-chain immutable storage provided by IPFS or other Layer 2 data availability layer storage. Alternatively, one could encrypt only one copy of the recipe, with the same recipe private key stored in the secure enclave of each hardware device.

Time-based one-time passwords can be used to reduce the chance of cracking the encryption. These use an algorithm to ensure that any password sent is valid only for a certain period. In this case, the controlling entity would need to rewrite the state 240 of the central smart contract 245 each time the one-time password is updated. The controlling entity's client 230 and the device 101 would share the specific algorithm updating the TOTP (time-based one-time password) as a secret. The decryption would be executed on the device in the secure enclave so that the main SoC 140 cannot access the secret keys. Another factor contributing to the overall security of the system 200 derives from the fact that that smart contracts are not deployed to the blockchain in clear text but in compiled bytecode for execution by the EVM. The compilation process provides a measure of security. EVM bytecode differs from object code produced by a traditional compiler for a physical processor in specific ways, which makes reverse engineering of EVM bytecode more challenging. Therefore, it is difficult for an adversary to reverse-engineer the functions contained in the two smart contracts 220 and 245.
1. In some embodiments, a device comprises a secure enclave at least one processor executing an application, the application when executed, causing the at least one processor to at least generate a request for one or more treatments to be executed by the treatment device, retrieve data specifying the one or more treatments from a blockchain or data availability source, request authorization for a selected one of the one or more treatments from the blockchain, and in response to receiving authorization from the blockchain, cause the device to execute the selected treatment based on data specifying the selected treatment.
2. The device of clause 1, further comprising an anti-tampering module configured to detect a tampering attempt in the treatment device.
3. The device of clauses 1 or 2, wherein the anti-tampering module comprises one or more sensors to detect a temperature or pressure change exceeding a respective threshold.
4. The device of any of clauses 1-3, wherein the anti-tampering module comprises one or more sensors to monitor abnormal voltage or frequency levels indicating interference with electrical operation of the treatment device.
5. The device of any of clauses 1-4, wherein the anti-tampering module comprises one or more light sensors that detect light exposure internal to the treatment device.
6. The device of any of clauses 1-5, wherein the application, when executed, causes the one or more processors to initiate erasure of a secure enclave upon receiving an indication of tampering from the anti-tampering module.
7. The device of any of clauses 1-6, wherein the application, when executed, causes the one or more processors to initiate an alarm upon receiving an indication of tampering from the anti-tampering module.
8. The device of any of clauses 1-7, wherein the application, when executed, causes the one or more processors to initiate a device shutdown upon receiving an indication of tampering from the anti-tampering module.
9. The device of any of clauses 1-8 wherein the secure enclave stores a private key used for signing and decrypting of messages from and to the device respectively and the contents of the secure enclave cannot be retrieved or revealed.
10. The device of any of clauses 1-9, a user authentication module, a blockchain client, and a transcranial ultrasound hardware used for ultrasound targeting and stimulation.
11. In some embodiments, a system comprises a treatment device, a control client and a physician client, wherein the treatment device, control client and physician client communicate with a first and a second smart contract in a network.
12. The system of clause 11, wherein the treatment device requests and receives a treatment list, treatment characteristics, and prices from the system, wherein the treatment list, treatment characteristics and prices are accessed through a second smart contract.
13. The system of clauses 11 or 12, wherein the treatment device executes a blockchain transaction that pays for the wellness treatment or prescribed treatment to system, and upon successful completion of the blockchain transaction the second smart contract is altered.
14. The system of any of clauses 11-13, wherein the treatment device requests and receives authorization from a first smart contract for a treatment of a user or patient.
15. The system of any of clauses 1-81 wherein the treatment device retrieves a treatment recipe from a second smart contract and performs the treatment on the user using the treatment recipe retrieved.
16. The system of any of clauses 11-15, wherein the control device modifies a second smart contract stored on the blockchain, the modification is associated with a cost of a treatment, a configuration of a treatment, or a treatment list.
17. The system of any of clauses 11-16, wherein the physician using a physician device executes a blockchain transaction with a first smart contract that creates a prescription treatment or an authorization for a user or patient.
18. The system of any of clauses 11-17, wherein the blockchain comprises Ethereum, and the authorization is provided via smart contract code running on the Ethereum Virtual Machine.
19. The system of any of clauses 11-18, wherein the blockchain comprises Solana, and the authorization is provided via smart contract code running in a Solana Virtual Machine.
20. The system of any of clauses 11-19, wherein the blockchain is a layer-2 chain settling to Ethereum.
21. In some embodiments, a method comprises a physician authorizing or creating a treatment plan for a user using a device in a blockchain network, the user's treatment device in the blockchain network further generating a request for a treatment list, receiving treatment list or prescribed treatment list for the user, requesting a prescription treatment recipe from the prescription treatment list, requesting and receiving authorization for the prescription treatment, sending a payment for the authorized prescription recipe, receiving the prescription treatment recipe and performing the treatment as per the treatment recipe.
22. In some embodiments, a method comprises of a treatment device of a user in a blockchain network requesting a treatment list and receiving treatment list from a blockchain network, requesting a treatment recipe from the treatment list, sending a payment to the blockchain network for the treatment recipe, receiving the wellness treatment recipe, and performing the treatment on the user as per the treatment recipe.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, for example, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A device comprising:
a secure enclave;
at least one processor executing an application, the application when executed,
causing the at least one processor to at least:
generate a request for one or more treatments to be executed by the treatment device;
retrieve data specifying the one or more treatments from a blockchain or data availability source;
request authorization for a selected one of the one or more treatments from the blockchain; and
in response to receiving authorization from the blockchain, cause the device to execute the selected treatment based on data specifying the selected treatment.

2. The device of claim 1, further comprising an anti-tampering module configured to detect a tampering attempt in the treatment device.

3. The device of claim 2, wherein the anti-tampering module comprises one or more sensors to detect a temperature or pressure change exceeding a respective threshold.

4. The device of claim 2, wherein the anti-tampering module comprises one or more sensors to monitor abnormal voltage or frequency levels indicating interference with electrical operation of the treatment device.

5. The device of claim 2, wherein the anti-tampering module comprises one or more light sensors that detect light exposure internal to the treatment device.

6. The device of claim 2, wherein the application, when executed, causes the one or more processors to initiate erasure of a secure enclave upon receiving an indication of tampering from the anti-tampering module.

7. The device of claim 2, wherein the application, when executed, causes the one or more processors to initiate an alarm upon receiving an indication of tampering from the anti-tampering module.

8. The device of claim 2, wherein the application, when executed, causes the one or more processors to initiate a device shutdown upon receiving an indication of tampering from the anti-tampering module.

9. The device of claim 1 wherein the secure enclave stores a private key used for signing and decrypting of messages from and to the device respectively and the contents of the secure enclave cannot be retrieved or revealed.

10. The device of claim 1 further comprising a communication module, a user authentication module, a blockchain client, and a transcranial ultrasound hardware used for ultrasound targeting and stimulation.

11. A system comprising:
a treatment device; a control client and
a physician client, wherein the treatment device, control client and physician client communicate with a first and a second smart contract in a network.

12. The system of claim 11, wherein the treatment device requests and receives a treatment list, treatment characteristics, and prices from the system, wherein the treatment list, treatment characteristics and prices are accessed through a second smart contract.

13. The system of claim 11, wherein the treatment device executes a blockchain transaction that pays for the wellness treatment or prescribed treatment to system; and upon successful completion of the blockchain transaction the second smart contract is altered.

14. The system of claim 11, wherein the treatment device requests and receives authorization from a first smart contract for a treatment of a user or patient.

15. The system of claim 11 wherein the treatment device retrieves a treatment recipe from a second smart contract and performs the treatment on the user using the treatment recipe retrieved.

16. The system of claim 11, wherein the control device modifies a second smart contract stored on the blockchain; the modification is associated with a cost of a treatment, a configuration of a treatment, or a treatment list.

17. The system of claim 11, wherein the physician using a physician device executes a blockchain transaction with a first smart contract that creates a prescription treatment or an authorization for a user or patient.

18. The system of claim 11, wherein the blockchain comprises Ethereum, and the authorization is provided via smart contract code running on the Ethereum Virtual Machine.

19. The system of claim 11, wherein the blockchain comprises Solana, and the authorization is provided via smart contract code running in a Solana Virtual Machine.

20. The system of claim 11, wherein the blockchain is a layer-2 chain settling to Ethereum.

21. A method comprising:
a physician authorizing or creating a treatment plan for a user using a device in a blockchain network;
the user's treatment device in the blockchain network further:
generating a request for a treatment list;
receiving treatment list or prescribed treatment list for the user;
requesting a prescription treatment recipe from the prescription treatment list;
requesting and receiving authorization for the prescription treatment;
sending a payment for the authorized prescription recipe; and
receiving the prescription treatment recipe and performing the treatment as per the treatment recipe.

22. A method comprising of a treatment device of a user in a blockchain network:
requesting a treatment list and receiving treatment list from a blockchain network;
requesting a treatment recipe from the treatment list;
sending a payment to the blockchain network for the treatment recipe;
receiving the wellness treatment recipe; and
performing the treatment on the user as per the treatment recipe.
